(19) United States
(12) Patent Application Publication
(10) Pub. No.: US 2013/0018343 A1
(43) Pub. Date: Jan. 17, 2013

(54) Lotions Derived From Renewable Resources and Absorbent Articles Comprising Same

(71) Applicants: Raphael Warren, Amberley Village, OH (US); Paul Thomas Weisman, Cincinnati, OH (US)

(72) Inventors: Raphael Warren, Amberley Village, OH (US); Paul Thomas Weisman, Cincinnati, OH (US)

(21) Appl. No.: 13/182,028

(22) Filed: Jul. 13, 2011

(51) Int. Cl. A61L 15/44; A61Q 19/00; A61P 17/00; A61Q 17/04; A61K 31/01; A61K 8/92

(52) U.S. Cl. 604/367; 514/762; 424/59

(57) ABSTRACT

A lotion having an emollient and an immobilizing agent. The emollient is derived from a renewable resource and having a bio-based content of from about 10% to about 100% using ASTM D6866-10, method B and the immobilizing agent selected from the group consisting of C₁₄-C₆₀ fatty alcohols, C₁₄-C₆₀ fatty acids, C₁₄-C₆₀ fatty alcohol ethoxylates having an average degree of ethoxylation ranging from about 2 to about 110, waxes and mixtures thereof. Absorbent articles comprising said lotions are also provided for herein.

### TECHNICAL FIELD

The present disclosure generally relates to lotions derived from renewable resources and absorbent articles comprising the same.

### BACKGROUND

Many products today require highly engineered components and yet, at the same time, these products are required to be limited use or disposable items. By limited use or disposable, it is meant that the product and/or component is used only a small number of times or possibly only once before being discarded. Examples of such products include, but are not limited to, absorbent articles such as diapers, training pants, incontinence garments, sanitary napkins, bandages, wipes, tissue-towel paper products. These types of products can and do utilize lotions. When lotions are used in limited use and/or disposable products, the impetus for reducing cost is extremely high.

Most of the materials used in current commercial lotions used in conjunction with absorbent articles are derived from non-renewable resources, such as petroleum. Typically, the components of lotions (e.g., emollients) are made from mineral oils or petrolatum, both of which are derived from petroleum based sources.

The price and availability of the petroleum feedstock ultimately has a significant impact on the price of lotions which utilize components derived from petroleum. As the worldwide price of petroleum escalates, so does the price of such lotions and absorbent articles utilizing such lotions.

Furthermore, many consumers display an aversion to purchasing products that are derived from petrochemicals. In some instances, consumers are hesitant to purchase products made from limited non-renewable resources such as petroleum and coal. Other consumers may have adverse perceptions about products derived from petrochemicals being “unnatural” or not environmentally friendly.

Accordingly, it would be desirable to provide a lotion for use in connection with absorbent articles which in part is derived from renewable resources. Accordingly, it would be desirable to provide a lotion for an absorbent article which reduces the use of petroleum and lowers costs, such that the lotion has improved performance characteristics to satisfy product needs.

### SUMMARY

In accordance with one embodiment, a lotion comprises an emollient and an immobilizing agent. The emollient is selected from the group consisting of mineral oil, petrolatum, and mixtures thereof. The emollient is derived from a renewable resource and has a bio-based content of from about 10% to about 100% using ASTM D6866-10, method B. The immobilizing agent is selected from the group consisting of C₁₄-C₆₀ fatty alcohols, C₁₄-C₆₀ fatty acids, C₁₄-C₆₀ fatty alcohol ethoxylates having an average degree of ethoxylation ranging from about 2 to about 110, waxes and mixtures thereof.

In accordance with another embodiment, a lotion comprises an emollient and an immobilizing agent. The lotion has a bio-based content of from about 10% to about 100% using ASTM D6866-10, method B. The emollient is selected from the group consisting of natural fats, natural oils, mineral oil, petrolatum, and mixtures thereof. The emollient has an Oil Stability Index of at least about 10 hours. The immobilizing agent is selected from the group consisting of C₁₄-C₆₀ fatty alcohols, C₁₄-C₆₀ fatty acids, C₁₄-C₆₀ fatty alcohol ethoxylates having an average degree of ethoxylation ranging from about 2 to about 110, waxes and mixtures thereof.

In accordance with yet another embodiment, an absorbent article comprises a topsheet, a lotion, a backsheet and an absorbent core. The lotion is applied to at least a portion of the topsheet. The lotion comprises an emollient having an Oil Stability Index of at least 10 hours. The lotion has a bio-based content of from about 10% to about 100% using ASTM D6866-10, method B. The absorbent core is disposed between the topsheet and the backsheet.

### BRIEF DESCRIPTION OF THE DRAWING

FIG. 1 is a top view of an absorbent article comprising a topsheet, a backsheet, and an absorbent core, with a lotion applied thereto.

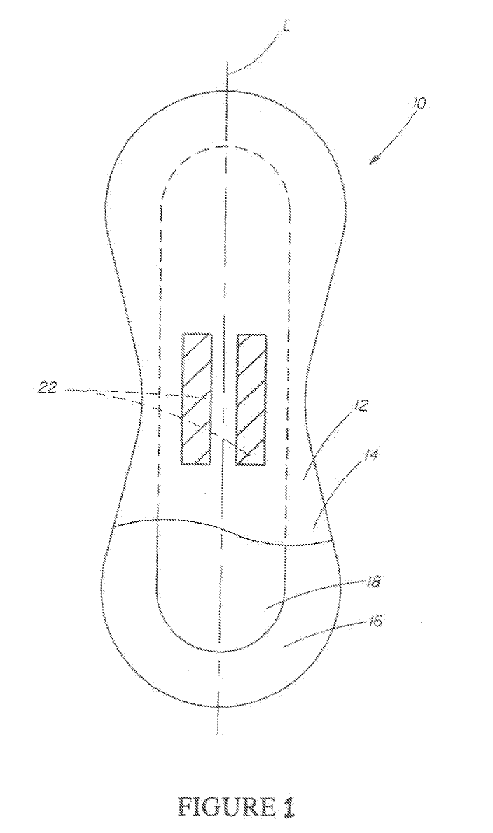

While the specification concludes with claims particularly pointing out and distinctly claiming the subject matter that is regarded as the present invention, it is believed that the invention will be more fully understood from the following description taken in conjunction with the accompanying drawing. FIG. 1 may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

### DETAILED DESCRIPTION

### I. Definitions

As used herein, the following terms shall have the meaning specified thereafter:

“Absorbent article” means devices that absorb and/or contain liquid. Wearable absorbent articles are absorbent articles placed against or in proximity to the body of the wearer to absorb and contain various exudates discharged from the body. Non-limiting examples of wearable absorbent articles include diapers, pant-like or pull-on diapers, training pants, sanitary napkins, tampons, panty liners, incontinence devices, and the like. Additional absorbent articles include wipes and cleaning products.

“Bio-based content” refers to the amount of carbon from a renewable resource in a material as a percent of the mass of the total organic carbon in the material, as determined by ASTM D6866-10, method B. Note that any carbon from inorganic sources such as calcium carbonate is not included in determining the bio-based content of the material.

“Disposed” refers to an element being located in a particular place or position.

“Disposable” refers to absorbent articles which are not intended to be laundered or otherwise restored or reused as an absorbent article after a single use.

“Emollient” refers a material that protects against wetness or irritation, softens, soothes, supples, coats, lubricates, moisturizes, protects and/or cleanses the skin.

“Flexible” refers to materials which are compliant and can readily conform to the general shape and contours of the human body.

“Petrochemical” refers to an organic compound derived from petroleum, natural gas, or coal.

“Petroleum” refers to crude oil and its components of paraffinic, cycloparaffinic, and aromatic hydrocarbons. Crude oil may be obtained from tar sands, bitumen fields, and oil shale.

“Related environmental message” refers to a message that conveys the benefits or advantages of the lotion and/or absorbent article formed from a renewable resource. Such benefits include being more environmentally friendly, having reduced petroleum dependence, being derived from renewable resources, and the like.

“Renewable resource” refers to a natural resource that can be replenished within a 100 year time frame. The resource may be replenished naturally, or via agricultural techniques. Renewable resources include plants, animals, fish, bacteria, fungi, and forestry products. They may be naturally occurring, hybrids, or genetically engineered organisms. Natural resources such as crude oil, coal, and peat which take longer than 100 years to form are not considered to be renewable resources.

### II. Lotion Compositions

Lotion compositions can be directed to maintain and/or improve the skin appearance and/or condition of the areas in contact with an absorbent article. In certain embodiments, the lotion can provide a protective, non-occlusive function (e.g., a relatively liquid impervious but vapor pervious barrier) to avoid skin overhydration and skin exposure to materials contained in body exudates; an abrasion minimizing function to reduce skin irritation in the areas where the absorbent article is in contact with the wearer's skin; or contains ingredients that deliver, either directly or indirectly, skin care benefits. For example, the direct benefits may be directed towards redness reduction or anti-inflammatory action. The indirect benefits may be directed towards removal or reduction of skin irritants in urine or feces, or reduction in overhydration of the skin. The lotion can contain emollients or other skin care actives that protect or improve the skin condition against chaffing, overhydration or itchiness. Furthermore, the lotion can have a smooth, silky, non-grainy skin feel to minimize abrasion of sensitive or compromised skin having chronic conditions such as chaffing, dryness, or rashes.

The lotion of the present disclosure can include a substantially anhydrous, oil-based carrier comprising an emollient and an immobilizing agent.

A. Emollients

For lotions designed to provide skin appearance and/or skin protective benefits, a useful ingredient in these lotion compositions is one or more emollients. In certain embodiments, these emollients will have either a plastic or liquid consistency at room temperatures, i.e., 20° C.

Suitable emollients can include those hydrocarbons, or mixtures of hydrocarbons, having chain lengths of from 16 to 50 carbon atoms. Hydrocarbons having these chain lengths include mineral oil (also known as “liquid petrolatum”) and petrolatum (also known as “mineral wax,” “petroleum jelly” and “mineral jelly”). Mineral oil usually refers to less viscous mixtures of hydrocarbons having from 16 to 20 carbon atoms. Petrolatum usually refers to more viscous mixtures of hydrocarbons having from 16 to 32 carbon atoms. Other suitable emollients include natural fats and natural oils. In certain embodiments the emollient can be selected from the group consisting of petrolatum, mineral oil and mixtures thereof. In certain embodiments the emollient includes from about 30% to about 90% petrolatum, by weight, and in other certain embodiments the emollient comprises from about 45% to about 85% petrolatum, by weight.

In certain embodiments the emollient can be derived from a renewable resource. For example, in certain embodiments, the emollient can include petrolatum formed from a renewable resource such as a natural gas source. Suitable petrolatum formed from a renewable resources is described in PCT Publication No. WO 2008/128232, including methods of making the same. Charcoal derived from biomass can also be used to create syngas (i.e., CO+H₂) from which hydrocarbons such as ethane and propane can be prepared (Fischer-Tropsch Process).

Other suitable emollients can include fatty acid ester type emollients, alkyl ethoxylate type emollients, fatty alcohol type emollients, and combinations thereof. Examples of each of these types of emollients (as well as others) are described in U.S. Pat. No. 6,570,054.

Other suitable emollients can include natural oils or fats, or natural oil or fat derivatives, in particular of plant or animal origin. Non-limiting examples include oleic canola Oil (Brassica campestris, B. napus, B. rapa; characterized by having an oleic content greater than 70%, e.g., hi oleic canola oil, very high oleic canola oil, or partially hydrogenated canola oil), manila kernel oil (Sclerocarya birrea), palm oil (Elaeis Guineensis Oil), palm olein, palm stearin, palm superolein, pecan oil, pumpkin seed oil, oleic safflower oil (Carthamus Tinctorius; characterized by having an oleic content of greater than about 30% and omega-6 fatty acid content of less than about 50%, e.g., hi oleic safflower oil), sesame oil (Sesamum indicum, S. oreintale), soybean oil (Glycine max, e.g., hi oleic soybean, low linolenic soybean oil, partially hydrogenated), oleic sunflower oil (Helianthus annus; characterized by having an oleic content of greater than about 40%, e.g., mid oleic sunflower or high oleic sunflower oil), apricot oil, babassu oil, castor oil, coconut oil, cod liver oil, hydrogenated corn oil, hydrogenated cottonseed oil, hazelnut oil, jojoba oil, macadamia oil, meadowfoam seed oil, mink oil, maring a oil, manila oil, mortierella oil, palm kernel oil, hydrogenated peanut oil, hydrogenated rapeseed oil, rose hip oil, hydrogenated safflower oil, hydrogenated soybean oil, hydrogenated sunflower oil, hydrogenated walnut oil, hydrogenated wheat germ oil, or the hardened derivatives thereof. Other non-limiting examples of fats and oils suitable skin care active options herein include: butter, C12-C18 acid triglyceride, caprylic/capric/lauric triglyceride, caprylic/capric/linoleic triglyceride, caprylic/capric/stearic triglyceride, caprylic/capric triglyceride, cocoa butter, C10-C18 triglycerides, egg oil, epoxidized soybean oil, glyceryl triacetyl hydroxystearate, glyceryl triacetyl ricinoleate, glycosphingolipids, hydrogenated castor oil, hydrogenated castor oil laurate, hydrogenated coconut oil, hydrogenated C12-C18 triglycerides, hydrogenated fish oil, hydrogenated lard, hydrogenated menhaden oil, hydrogenated mink oil, hydrogenated orange roughy oil, hydrogenated shark liver oil, hydrogenated tallow, hydrogenated vegetable oil, lanolin and lanolin derivatives, lanolin alcohol, lard, lauric/palmitic/oleic triglyceride, lesquerella oil, maleated soybean oil, neatsfoot oil, oleic/linoleic triglyceride, oleic/palmitic/lauric/myristic/linoleic triglyceride, oleostearine, olive husk oil, omental lipids, pengawar djambi oil, pentadesma butter, phospholipids, shea butter, tallow, tribehenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, and the like, as well as mixtures thereof. Oleic canola oil, palm oil, sesame oil, hi oleic safflower oil, hi oleic soybean oil, mid oleic sunflower oil, and high oleic sunflower oil are common plant-bred derived oils and may be also be derived from non-genetically modified organisms (non-GMO). Additional such emollients are also described in U.S. patent application Ser. No. 12/974,674.

In certain embodiments, the emollient can further comprise a blend of oils, including those described supra, as well as additional oil materials. Suitable additional emollients can include acai berry oil, almond oil, avocado oil, beech oil, brazil nut oil, camelina sativa oil (family Brassicaceae, e.g. Camelina Sativa, Gold of Pleasure, False Flax, etc.), camellia seed oil, canola oil, carrot seed oil, cashew nut oil, caster oil, cherry kernel oil, chia oil, corn oil, cottonseed oil, hydrogenated cottonseed oil, evening primrose oil, filbert (hazelnut) oil, grapeseed oil, hemp oil, hickory nut oil, jojoba oil, kukui oil, lanolin, olive oil (Olea europaea), macadamia oil, maring a oil, meadowfoam oil, neem oil, palm kernel oil, olive oil, passionflower oil (family Passiflora, Passiflora Incarnata), peanut oil, peach kernel oil, pistachio nut oil, rapeseed oil, rice bran oil, rose hip oil, safflower oil, sorghum oil, soybean oil, sunflower seed oil, tall oil, vegetable oil, vegetable squalene, walnut oil, wheat germ oil, and mixtures thereof. The oil material of the present invention can be selected from the group consisting of camelina sativa seed oil, oleic canola oil, evening primrose oil, manila kernel oil, palm oil, palm olein, palm stearin, palm superolein, passiflora incarnata seed oil, pecan oil, pumpkin seed oil, oleic safflower oil, sesame oil, soybean oil, oleic sunflower oil, vegetable oil and mixtures thereof.

In certain embodiments, to further enhance the stability of the emollient, certain antioxidants can be added to certain emollients or to the lotion composition. In one embodiment, the emollient comprises from about 0.005% to about 1%, from about 0.01% to about 0.5%, or from about 0.02% to about 0.2%, by weight of the emollient, of an antioxidant. In one embodiment, the lotion composition comprises from about 0.0005% to about 1%, from about 0.001% to about 0.75%, or from about 0.002% to about 0.5%, by weight of the lotion composition, of an antioxidant. Non-limiting examples of suitable antioxidants include α-tocopherol, β-tocopherol, γ-tocopherol, δ-tocopherol, tocotrienol, rosemary, sesamol, sesamolin, sesamin, catechin, and mixtures thereof.

B. Immobilizing Agents

Another component of the lotion is an agent capable of immobilizing the composition in the desired location in or on the treated article. Because some embodiments of the composition have a plastic or liquid consistency at 20° C., they tend to flow or migrate, even when subjected to modest shear. When applied to a wearer-contacting surface or other location of an absorbent article, especially in a melted or molten state, the lotion composition will not remain primarily in or on the treated region. Instead, the lotion composition will tend to migrate and flow to undesired regions of the article and adversely affect the absorbency of the article.

Specifically, if the lotion composition migrates into the interior of the absorbent article, it can cause undesired effects on the absorbency of the absorbent article. It also means that much more of the lotion has to be applied to the article to get the desired skin smoothness benefits. Increasing the add-on level of lotion composition not only increases the cost, but also exacerbates the undesirable effect on the absorbency of the article's core and undesired transfer of lotion during processing/converting of the treated articles.

The immobilizing agent counteracts this tendency of the lotion (without the immobilizing agent) to migrate or flow by keeping the composition primarily localized on the surface or in the region of the article to which the lotion is applied. This is believed to be due, in part, to the fact that the immobilizing agent raises the melting point and/or viscosity of the lotion composition. Since the immobilizing agent can be miscible with the emollient (or solubilized in the emollient with the aid of an appropriate emulsifier or dispersed therein), it entraps the lotion on the surface of the absorbent article's wearer contacting surface or in the region to which it is applied.

In addition to being miscible with (or solubilized in) the emollient, the immobilizing agent can have a melting profile that will provide a lotion composition that is solid or semisolid at room temperature. In this regard, certain embodiments of immobilizing agents can have a melting point of at least about 35° C. This is so the immobilizing agent itself will not have a tendency to migrate or flow. In other certain embodiments, the immobilizing agent can have melting points of at least about 40° C. In still other certain embodiments, the immobilizing agent can have a melting point in the range of from about 50° to about 150° C.

Suitable immobilizing agents can be selected from the group consisting of C₁₄-C₆₀ fatty alcohols, C₁₄-C₆₀ fatty acids, C₁₄-C₆₀ fatty alcohol ethoxylates having an average degree of ethoxylation ranging from about 2 to about 110, waxes and mixtures thereof. The “average degree of ethoxylation” refers to the number of units of ethoxylates. In certain embodiments, such immobilizing agents can include C₁₆-C₂₂ fatty alcohols selected from the group consisting of cetyl alcohol, stearyl alcohol, behenyl alcohol, and mixtures thereof. Other suitable immobilizing agents include, either alone, or in combination with the above-mentioned immobilizing agents, waxes such as carnauba, ozokerite, beeswax, candelilla, paraffin, ceresin, esparto, ouricuri, rezowax, isoparaffin, and other known mined and mineral waxes. The high melt point of these materials can help immobilize the composition on the desired surface or location on the article. Additionally microcrystalline waxes are effective immobilizing agents. Examples of these and other immobilizing agents are described in U.S. Pat. No. 6,570,054.

C. Surfactants

In certain embodiments surfactants can be added to such lotions as described herein. Such surfactants can be miscible with the other components of the lotion composition so as to form blended mixtures (e.g., hydrophilic surfactants). Because of possible skin sensitivity of those using disposable absorbent products to which the composition is applied, these surfactants should also be relatively mild and non-irritating to the skin. Typically, these hydrophilic surfactants are nonionic to be not only non-irritating to the skin, but also to avoid other undesirable effects on any other structures within the treated article. For example, reductions in tissue laminate tensile strength, adhesive bond sufficiencies, and the like.

Suitable nonionic surfactants may be substantially nonmigratory after the lotion is applied to the absorbent articles and will typically have HLB values in the range of from about 4 to about 20, and from about 7 to about 20. To be nonmigratory, such nonionic surfactants can typically have melt temperatures greater than the temperatures commonly encountered during storage, shipping, merchandising, and use of disposable absorbent products, e.g., at least about 30° C. In this regard, these nonionic surfactants will preferably have melting points similar to those of the immobilizing agents previously described. Examples of such suitable surfactants are described in U.S. Pat. No. 6,570,054.

D. Rheological Agents

Rheological agents can be added to the lotion composition to suspend the lotion components and maintain a stable suspension. The carrier (e.g., the emollient and immobilizing agent) without the rheological agents exhibits typical Newtonian fluid characteristics, that is, the dispersed particles, upon standing, frequently agglomerate and separate from the carrier. This drawback can lead to settling and bridging effects during processing and failure to apply the skin care composition to a substrate surface consistently. The rheology of the composition in its melt phase may be modified by an effective amount of the rheological agent(s) such that it behaves like a plastic or pseudoplastic fluid. The resultant lotion composition is a stable solution or suspension having finely dispersed skin care components therein. The stabilized composition is substantially free of agglomeration, stratification and/or settling; therefore, the melt composition can flow through processing equipment easily and be consistently applied to a substrate surface. It is found that both the elastic modulus and the apparent viscosity of the composition are factors affecting the processability of the lotion composition.

Specifically, the addition of a rheological agent can increase the elastic modulus of the lotion composition in certain embodiments to at least about 5 dyne/cm² when measured at 77° C. under an oscillation frequency of 10 rad/sec and a shear strain of 0.2% (see test method disclosed herein). In other certain embodiments, the lotion composition can have an elastic modulus in the range from about 5 to about 25,000 dyne/cm²; from about 10 to about 10,000 dyne/cm²; and from about 100 to about 5,000 dyne/cm². Examples of such rheological agents are described in U.S. Pat. No. 6,570,054.

The rheological properties (such as elastic modulus, viscosity) of the lotion in the melt form are measured using a viscometer (available from TA Instruments of New Castle, Del. as model number CSL 100) in an oscillation mode. The measurements are conducted using a cone-and-plate measuring system, having a diameter of 40 mm and a gap of 60 micron. The measurement commences after about 100 seconds waiting time. And the measurements are conducted at two temperatures: 77° C. and 40° C. The elastic modulus measured at 10 rad/sec frequency and 0.2% strain is used to characterize the compositions. That is, all the elastic moduli disclosed and/or claimed herein are measured at the operating conditions given above.

Certain embodiments of the lotion compositions can be solid, or more often semi-solid at room temperature, i.e., at 20° C. Being solid or semi-solid at room temperature, the lotions do not have a tendency to flow and migrate to a significant degree to undesired locations of the article, and thus avoid significant interference with the absorbency of the article. This means less lotion is required for imparting desirable appearance, protective or conditioning benefits. In certain embodiments, lotions of the present disclosure can have a zero shear viscosity at about 20° C. between about 1.0×10⁶ centipoise and about 1.0×10⁸ centipoise; in certain embodiments from between about 5.0×10⁶ centipoise and about 5.0×10⁷ centipoise; and in certain embodiments from between about 7.0×10⁶ centipoise and about 1.0×10⁷ centipoise. Generally, the value for “zero shear viscosity” can be obtained by extrapolating a viscosity versus shear rate plot to a shear rate of zero. However, for plastic or pseudoplastic fluids which exhibit a yield behavior at low shear rate, the extrapolation method often does not fully and accurately describe the material. Alternatively, the “zero shear viscosity” can be approximated by a viscosity measured at very low shear rates. As used herein, the term “zero shear viscosity” is the value measured by a cone and plate viscometer (available from TA Instruments of New Castle, Del. as model number CSL 100), at very low shear rates (e.g., 1.0 sec⁻¹ or lower) and at a temperature of about 20° C.

E. Optional Skin Care Actives

In certain embodiments, the lotion may contain at least one skin care active. Such skin care actives may be insoluble or partially soluble solids in the substantially anhydrous, oil-based carrier. The skin care actives may be incorporated into the lotion composition, either directly or as a predispersion, with agitation.

Such skin care actives can include, but are not limited to, proton donating agents, protease and/or enzyme inhibitors, antimicrobials, humectants (glycerine, sorbitol), vitamins and derivatives thereof (e.g., Vitamins A, D, E and K), skin soothing and healing agents, such as aloe vera, or other ingredients from herbal, botanical or mineral sources, sunscreens, preservatives, anti-acne medicaments, antioxidants, chelators and sequestrants, essential oils, skin sensates, multi-functional agents, such as zinc oxide, and mixtures thereof. Examples of such skin care actives are described in U.S. Pat. No. 6,570,054 and U.S. patent application Ser. No. 12/974,674.

### III. Hardness Properties of Lotions

The hardness of the lotions of this present disclosure can be important for at least two reasons. First, the softer the formulation the more mobile the formulation will be, making the formulation more likely to migrate, which is not desirable. Secondly, softer lotions tend to be more greasy/oily to the touch, which is also less desirable. In general, lotions having a needle penetration hardness of from about 200 to about 365 millimeters feel creamy to slightly greasy with less smoothness (depending on additives). Lotions that have needle penetration hardness values of from about 5 to about 200 millimeters feel silky to creamy and very smooth (depending on additives). Certain embodiments of penetration hardness of the lotions can be from about 5 to about 365 millimeters, from about 10 to about 300 millimeters, from about 20 to about 200 millimeters, or from about 40 to about 120 millimeters. Lotion compositions having a needle penetration hardness between about 5 and 365 millimeters can be measured using ASTM method D 1321.

### IV. Oxidative Properties of Lotions

Unsaturated fatty acids tend to be instable and tend to easily oxidize. Oxidation can be promoted by multiple sources that include temperature, light, air, oxygen, moisture, and metals. See, e.g., Belitz H-D, Grosch W, and Schieberle P, Lipids In Food Chemistry 3^{rd} ed. Springer-Verlag, Heidelberg, 2004, p. 157-242. Indeed, common sources of product making can promote instability. For example, melting and mixing the ingredients to form a lotion can require high temperatures (to a temperature above the melting point of the ingredients for the lotion, e.g., greater than 70° C.). In order to melt and preserve the uniformity of a semi-solid lotion, it is common to heat the lotion application tank to high temperatures (e.g., greater than 60° C., preferably above 70° C.) with mixing. Furthermore, the lotion can remain in the tank for a considerable amount of time (e.g., greater than 24 hours). Another source of instability can be the shelf storage of the finished product. It is not unusual for product to remain on the shelf (in the store or at home) for at least a year and, depending on geographical location, storage temperatures can exceed 40° C. Another source of instability can result from lotions that are water- or glycol-based. Collectively, these factors can lead to oxidation and creation of reactive oxygen-free radicals or active oxygen. This can lead to product deterioration such as discoloration (i.e., yellowing) and/or rancid odor. When in contact with the skin, active oxygen can damage skin barrier function.

A common measure for monitoring oxidative stability is the development of hydroperoxides (peroxide value or PV) over time. Oxidative stability can also be expressed in terms of the time required to obtain secondary oxidation products when aerating a sample at elevated temperature. A suitable measure of oxidative stability is called the Oil Stability Index (referred to herein as “OSI”). The OSI of an oil material can be measured according to the American Oil Chemical Soceity Oil Stability Index Method (AOCS Official Method Cd 12b-92).

In certain embodiments, the oil material used in the lotion described in the present disclosure can be selected to have an OSI of at least about 10 hours; in certain embodiments at least about 14 hours; and in certain embodiments at least about 18 hours.

### V. Validating Lotions Derived from Renewable Resources

A suitable validation technique is through ¹⁴C analysis. A small amount of the carbon dioxide in the atmosphere is radioactive. This ¹⁴C carbon dioxide is created when nitrogen is struck by an ultra-violet light produced neutron, causing the nitrogen to lose a proton and form carbon of molecular weight 14 which is immediately oxidized to carbon dioxide. This radioactive isotope represents a small but measurable fraction of atmospheric carbon. Atmospheric carbon dioxide is cycled by green plants to make organic molecules during photosynthesis. The cycle is completed when the green plants or other forms of life metabolize the organic molecules, thereby producing carbon dioxide which is released back to the atmosphere. Virtually all forms of life on Earth depend on this green plant production of organic molecules to grow and reproduce. Therefore, the ¹⁴C that exists in the atmosphere becomes part of all life forms, and their biological products. In contrast, fossil fuel based carbon does not have the signature radiocarbon ratio of atmospheric carbon dioxide.

Assessment of the renewably based carbon in a material can be performed through standard test methods. Using radiocarbon and isotope ratio mass spectrometry analysis, the bio-based content of materials can be determined. ASTM International, formally known as the American Society for Testing and Materials, has established a standard method for assessing the bio-based content of materials. The ASTM method is designated ASTM D6866-10.

The application of ASTM D6866-10 to derive a “bio-based content” is built on the same concepts as radiocarbon dating, but without use of the age equations. The analysis is performed by deriving a ratio of the amount of organic radiocarbon (¹⁴C) in an unknown sample to that of a modern reference standard. The ratio is reported as a percentage with the units “pMC” (percent modern carbon).

The modern reference standard used in radiocarbon dating is a NIST (National Institute of Standards and Technology) standard with a known radiocarbon content equivalent approximately to the year AD 1950. AD 1950 was chosen since it represented a time prior to thermo-nuclear weapons testing which introduced large amounts of excess radiocarbon into the atmosphere with each explosion (termed “bomb carbon”). The AD 1950 reference represents 100 pMC.

“Bomb carbon” in the atmosphere reached almost twice normal levels in 1963 at the peak of testing and prior to the treaty halting the testing. Its distribution within the atmosphere has been approximated since its appearance, showing values that are greater than 100 pMC for plants and animals living since AD 1950. It's gradually decreased over time with today's value being near 107.5 pMC. This means that a fresh biomass material such as corn could give a radiocarbon signature near 107.5 pMC.

Combining fossil carbon with present day carbon into a material will result in a dilution of the present day pMC content. By presuming 107.5 pMC represents present day biomass materials and 0 pMC represents petroleum derivatives, the measured pMC value for that material will reflect the proportions of the two component types. A material derived 100% from present day soybeans would give a radiocarbon signature near 107.5 pMC. If that material was diluted with 50% petroleum derivatives, for example, it would give a radiocarbon signature near 54 pMC (assuming the petroleum derivatives have the same percentage of carbon as the soybeans).

A biomass content result is derived by assigning 100% equal to 107.5 pMC and 0% equal to 0 pMC. In this regard, a sample measuring 99 pMC will give an equivalent bio-based content value of 92%.

Assessment of the materials described herein was done in accordance with ASTM

D6866. The mean values quoted in this report encompasses an absolute range of 6% (plus and minus 3% on either side of the bio-based content value) to account for variations in end-component radiocarbon signatures. It is presumed that all materials are present day or fossil in origin and that the desired result is the amount of bio-based component “present” in the material, not the amount of bio-based material “used” in the manufacturing process.

Emollients derived from renewable resources can have a bio-based content of from about 10% to about 100% using ASTM D6866-10, method B; certain embodiments the emollient can have a bio-based content of from about 30% to about 90% using ASTM D6866-10, method B; and certain embodiments the emollient can have bio-based content of from about 45% to about 85% using ASTM D6866-10, method B. In certain embodiments, the lotion composition can have a bio-based content of from about 10% to about 100% using ASTM D6866-10, method B; certain embodiments the lotion can have a bio-based content of from about 30% to about 90% using ASTM D6866-10, method B; and certain embodiments the lotion can have bio-based content of from about 45% to about 85% using ASTM D6866-10, method B.

In order to apply the methodology of ASTM D6866-10 to determine the bio-based content of a lotion, a representative sample of the lotion must be obtained for testing. For example, a sample of the lotion or emollient can be obtained prior to being added to the absorbent article. In an alternative embodiment, a representative amount of the lotion or emollient can be obtained from the absorbent article utilizing known separation techniques.

### VI. Absorbent Articles

As noted herein the lotions described herein can be applied to a variety of absorbent articles. Such absorbent articles can include diapers, training pants, incontinence garments, sanitary napkins, bandages, wipes, tissue-towel paper products, and any other suitable absorbent articles. It is important to note that the absorbent article also can be derived from a renewable resource. Examples of such absorbent articles are described in U.S. Patent Publication No. 2007/0219521.

FIG. 1 illustrates an exemplary absorbent article 10, that can be a sanitary napkin or pantiliner, having a body facing surface 12 comprising a topsheet 14, a backsheet 16 joined to the topsheet 14, and an absorbent core 18. The absorbent article 10 can have a longitudinal axis “L” and may also be provided with additional features commonly found in napkins, including “wings” or “flaps” (not shown) as is known in the art and/or a fluid acquisition layer to promote fluid transport to the absorbent core 18. Likewise, a topsheet of an absorbent article can have various optional characteristics, as is known in the art. For example, the topsheet 14 can have channels embossed therein to directed fluid flow, and can have apertures therethrough to aid in fluid acquisition. The topsheet 14 of the absorbent article 10 of the present disclosure can have a lotion 22 disposed onto the topsheet 14.

In certain embodiments, for example when an absorbent article is a sanitary napkin, the topsheet can be configured to be compliant, soft feeling and non-irritating to the wearers skin and hair. Further, the topsheet can be liquid pervious, permitting liquids (e.g., menses and/or urine) to readily penetrate through its thickness. A suitable topsheet may be manufactured from a wide range of materials such as woven an nonwoven materials (e.g, a nonwoven web of fibers); polymeric materials such as apertured formed thermoplastic films, apertured plastic films, and hydroformed thermoplastic films; porous foams; reticulated foams; reticulated thermoplastic films; and thermoplastic scrims. Suitable woven and nonwoven materials can be comprised of natural fibers (e.g., wood or cotton fibers), synthetic fibers (e.g., polymeric fibers such as polyester, polypropylene, or polyethylene fibers) or from a combination of natural and synthetic fibers. When the topsheet comprises a nonwoven web, the web may be manufactured by a wide number of known techniques. For example, the web may be spunbonded, carded, wet-laid, meltblown, hydroentangled, combinations of the above, or the like.

The backsheet of such an embodiment can be impervious to liquids (e.g., menses and/or urine) and can be manufactured from a thin plastic film, although other flexible impervious materials may also be used. The backsheet can prevent exudates absorbed and contained in the absorbent core from wetting articles which contact the absorbent article such as bedsheets, pants, pajamas and undergarments. The backsheet may thus comprise a woven or nonwoven material, polymeric films such as thermoplastic films of polyethylene or polypropylene, or composite materials such as film-coated nonwoven material. In one embodiment, the backsheet can be a breathable backsheet such as that described in U.S. Pat. No. 6,623,464.

The topsheet and backsheet can be positioned adjacent a body surface and a garment surface, respectively, of the absorbent core, such that the absorbent core is disposed between the topsheet and the backsheet. The absorbent core can be jointed with the topsheet, the backsheet, or both in any manner as is known by attachment means (not shown in FIG. 1) such as those known in the art. However, embodiments of the present disclosure are envisioned wherein portions of the entire absorbent core are unattached to either the topsheet, the backsheet, or both.

An absorbent article (e.g., sanitary napkin) of the present disclosure can have a low flexure-resistance, thus making the absorbent article highly flexible. In certain embodiments, the absorbent article can have a flexure-resistance of less than about 250.0 grams, in certain embodiments less than about 175.0 grams, and in certain embodiments less than about 130.0 grams. Examples of such absorbent articles are described in U.S. Pat. No. 5,951,537.

The flexure-resistance of an absorbent article (e.g., sanitary napkin) can be measured by peak bending stiffness. Peak bending stiffness can be determined by a test which is modeled after the ASTM D 4032-82 CIRCULAR BEND PROCEDURE, the procedure being considerably modified and performed as follows. The CIRCULAR BEND PROCEDURE is a simultaneous multi-directional deformation of a material in which one face of a specimen becomes concave and the other face becomes convex. The CIRCULAR BEND PROCEDURE gives a force value related to flexure-resistance, simultaneously averaging stiffness in all directions.

The apparatus necessary for the CIRCULAR BEND PROCEDURE can be a modified Circular Bend Stiffness Tester, having the following parts: A smooth-polished steel plate platform which is 102.0×102.0×6.35 millimeters having an 18.75 millimeter diameter orifice. The lap edge of the orifice should be at a 45 degree angle to a depth of 4.75 millimeters; a plunger having an overall length of 72.2 millimeters, a diameter of 6.25 millimeters; and a ball nose having a radius of 2.97 millimeters and a needle-point extending 0.88 millimeter therefrom having a 0.33 millimeter base diameter and a point having a radius of less than 0.5 millimeter, the plunger being mounted concentric with the orifice and having equal clearance on all sides. Note that the needle-point is merely to prevent lateral movement of the test specimen during testing. Therefore, if the needle-point significantly adversely affects the test specimen (for example, punctures an inflatable structure), than the needle-point should not be used. The bottom of the plunger should be set well above the top of the orifice plate. From this position, the downward stroke of the ball nose is to the exact bottom of the plate orifice. A force-measurement gauge and more specifically an Instron inverted compression load cell. The load cell has a load range of from about 0.0 to about 2000.0 grams. An actuator, and more specifically the Instron Model No. 1122 having an inverted compression load cell. The Instron 1122 is made by the Instron Engineering Corporation, Canton, Mass.

### Number and Preparation of Specimens

In order to perform the procedure for this test, as explained below, five representative sanitary napkins are necessary. From one of the five napkins to be tested, some number “Y” of 37.5.times.37.5 millimeter test specimens are cut. Specimens having portions in which a topsheet is joined directly to a barrier sheet or which are a laminate of a topsheet, two or less tissue sheets and a barrier sheet, should not be tested. The reason that these specimens are not tested is due to the realization that prior art napkins exist in which a topsheet is joined to a barrier sheet beyond the edges of an absorbent core in the periphery of the napkin, such portions of which are highly flexible. However, the present invention is more concerned with the overall flexibility of the sanitary napkin and not merely the peripheral portions thereof and, therefore, the flexibility of the present invention is more concerned with the flexibility of the significant absorbent portions of the sanitary napkin. If any of these significant absorbent portions of the sanitary napkin meet the parameters of this test, then the sanitary napkin satisfies the test. Therefore, a number of different specimens should be tested from each sanitary napkin. Certainly, the structurally most flexible portion of the sanitary napkin should be tested, excluding those portions excluded above. The test specimens should not be folded or bent by the test person, and the handling of specimens must be kept to a minimum and to the edges to avoid affecting flexural-resistance properties. From the four remaining sanitary napkins, an equal number “Y” of 37.5.times.37.5 millimeter specimens, identical to the specimens cut from the first napkin, are cut. Thus, the test person should have “Y” number of sets of five identical specimens.

### Procedure

The procedure for the CIRCULAR SEND PROCEDURE is as follows. The specimens are conditioned by leaving them in a room which is 21.+−0.1.degree. C. and 50.+−0.2% relative humidity for a period of two hours. The test plate is leveled. The plunger speed is set at 50.0 centimeters per minute per full stroke length. A specimen is centered on the orifice platform below the plunger such that the body surface 26 of the specimen is facing the plunger and the garment surface 17 of the specimen is facing the platform. The indicator zero is checked and adjusted, if necessary. The plunger is actuated. Touching the specimen during the testing should be avoided. The maximum force reading to the nearest gram is recorded. The above steps are repeated until all five of the identical specimens have been tested.

### Calculations

The peak bending stiffness for each specimen is the maximum force reading for that specimen. Remember that “Y” number of sets of five identical specimens were cut. Each set of five identical specimens is tested and the five values received for that set are averaged. Thus, the test person now has an average value for each of the “Y” sets tested. Remember, if any of the significantly absorbent portions of the sanitary napkin have the requisite flexure-resistance, then the napkin satisfies the parameters of this test. Therefore, the flexure-resistance for a particularly designed sanitary napkin is the greatest, of these average peak bending stiffnesses.

In certain embodiments, an absorbent article as described herein can have a free absorbent capacity of from about 4 grams per gram to about 125 grams per gram; in certain embodiments from about 10 grams per gram to about 100 grams per gram; and in certain embodiments from about 20 grams per gram to about 60 grams per gram. The test for measuring free absorbent capacity is discussed herein.

The test and free absorbent capacities of an absorbent article are determined as follows. Any adhesive release paper is removed from the absorbent article to be tested. To determine test capacity, a 4.75×14.0 centimeters portion of the absorbent article is cut from the portion of the absorbent article which would typically receive material to be absorbed. Free absorbent capacity is determined using the entire absorbent article minus any release paper. The article is weighed to the nearest 0.1 gram. The article is then submerged in a beaker of sterile saline (obtainable from the Baxter Travenol Company of Deerfield, Ill.), such that the article is totally submerged and is not bent or otherwise twisted or folded. The article is submerged for 10 minutes. The article is removed from the saline and suspended for two minutes in a vertical position to allow the saline to drain out of the article. The article is then placed body facing surface down onto an absorbent blotter, such as the filter paper #631 available from the Filtration Science Corp., Eaton-Dikeman Division of Mount Holly Springs, Pa. A uniform 17.6 grams per square centimeter load is placed over the article to squeeze excess fluid out. The absorbent blotter is replaced every 30 seconds until the amount of fluid transferred to the absorbent blotter is less than 0.5 grams in a 30 second period. Next, the article is weighed to the nearest 0.1 gram and the dry weight of the article is subtracted. The difference in grams is the test or free absorbent capacity of the article, whichever the case may be. Similar test is described in U.S. Pat. No. 5,951,537.

Because of the flexibility requirements of the absorbent articles, it is likely that the absorbent articles can be relatively thin. In certain embodiments, keeping the absorbent articles thin can give them a low wearing awareness and also keeps them as unobtrusive as possible. In certain embodiments, the caliper of an absorbent article can be less than about 10 mm; in certain embodiments less than about 8 mm; in certain embodiments from about 1 mm to about 5 mm; and in certain embodiments; from about 1.5 mm to about 4 mm. Such sanitary napkins 10 as shown in FIG. 1 can have a caliper of about 1.9 millimeters. The caliper of an absorbent article can be determined by the following test.

A comparator gauge, and specifically the Ames, Model 130 with dials indicator Model 482, available from the B.C. Ames, Company of Waltham, Mass. is needed. The comparator gauge should have a circular comparator foot made of aluminum and having a weight of 10.0 grams and a contact surface of 5.16 square centimeters. The comparator gauge is zeroed. An 80.0 grams stainless steel weight is placed on the spindle extending above the comparator dial. The comparator foot is raised and the absorbent article, with any adhesive release paper being removed, is placed down on the base plate. The absorbent article is positioned on the base plate so that when the foot is lowered it is in the center of the absorbent article. Try to smooth out or avoid any wrinkles in the absorbent article. Gently lower the foot onto the absorbent article. Determine the absorbent article caliper by reading the comparator dial 30 seconds after the foot comes in contact with the absorbent article. Repeat the measurement 3.0 centimeters from each of the ends of the absorbent material along a longitudinal centerline of the absorbent article. The average of the three readings is the caliper of the absorbent article. Similar test is described in U.S. Pat. No. 5,951,537.

### VII. Treating Absorbent Articles with Lotions

In preparing absorbent articles as described herein, the lotion composition can be applied such that during wear, at least some portion of the lotion composition will transfer from the treated article to the wearer's skin. That is, the lotion composition can either be applied directly to one or more wearer contacting surfaces, or can be applied in alternate locations or means such that the skin care composition is readily available for transfer from one or more wearer contacting surfaces during use without intervention by the user/caregiver. For example, materials positioned beneath the wearer contacting surface, encapsulated compositions, etc. Additionally, the lotion composition may be applied to other article regions for delivery to one or more of the wearer's hips, abdomen, back, waist, sides, thighs, etc. Nonlimiting examples of suitable methods include spraying, printing (e.g., flexographic printing), coating (e.g., contact slot coating, gravure coating), dipping, extrusion, or combinations of these application techniques, e.g. spraying the skin care composition on a rotating surface, such as a calender roll, then transfers the composition to the desired portion of the article. Alternatively, the skin care composition may be applied to a substrate as a solid or semi-solid material via a variety methods. It is to be understood that different application techniques/equipment are suited for materials with theological properties (e.g., shear viscosity, elastic modulus) in a particularly range. Other suitable techniques for treating absorbent articles with lotion compositions are described in U.S. Pat. No. 6,570,054.

In certain embodiments, the absorbent article comprises from about 2 mg to about 300 mg, in certain embodiments from about 5 mg to about 200 mg; and in certain embodiments from about 10 mg to about 150 mg of the lotion per absorbent article. The lotion can be applied to a portion of the absorbent article from about 2 gsm to about 100 gsm; from about 5 gsm to about 70 gsm, and from about 10 gsm to about 60 gsm. GSM or grams per meter squared is derived from the mass of lotion divided by the area on which the lotion is applied. The lotion can be applied to the topsheet of the absorbent article. The lotion composition can applied to the absorbent article in various defined patterns such as dot(s), stripe(s), square(s), circle(s), or oval(s). When applied as a stripe, the stripe length can be up to the length of the absorbent article and the width can be from about 0.1 mm to 50 mm; from about 0.5 mm to about 20 mm; and from about 1 mm to about 10 mm. To achieve a desirable benefit to the wearer, the lotion composition can be applied to specific regions of the absorbent article not limited to the longitudinal outer edge of the absorbent article, the area opposite the vaginal opening, or one end, or both ends of the absorbent article. For example, see European Application No. 1455716 and PCT Application No. WO2003/051260.

### VIII. Communicating a Related Environmental Message a Consumer

The present disclosure relating to absorbent articles incorporating lotions derived from renewable resources, further provides means for which to communicate an environmental message to a consumer. Such messages could be displayed on the absorbent article (or related packaging). The related environmental message may identify the absorbent article and/or lotion as: being environmentally friendly or Earth friendly; having reduced petroleum (or oil) dependence or content; having reduced foreign petroleum (or oil) dependence or content; having reduced petrochemicals or having components that are petrochemical free; and/or being made from renewable resources or having components made from renewable resources. This communication is of importance to consumers that may have an aversion to petrochemical use (e.g., consumers concerned about depletion of natural resources or consumers who find petrochemical based products unnatural or not environmentally friendly) and to consumers that are environmentally conscious. Without such a communication, the benefit of the present disclosure may be lost on some consumers.

The communication may be effected in a variety of communication forms. Suitable communication forms include store displays, posters, billboard, computer programs, brochures, package literature, shelf information, videos, advertisements, internet web sites, pictograms, iconography, or any other suitable form of communication. The information could be available at stores, on television, in a computer-accessible form, in advertisements, or any other appropriate venue. Ideally, multiple communication forms may be employed to disseminate the related environmental message.

The communication may be written, spoken, or delivered by way of one or more pictures, graphics, or icons. For example, a television or internet based-advertisement may have narration, a voice-over, or other audible conveyance of the related environmental message. Likewise, the related environmental message may be conveyed in a written form using any of the suitable communication forms listed above. In certain embodiments, it may be desirable to quantify the reduction of petrochemical usage of the present lotion compositions compared to lotion compositions that are presently commercially available.

The related environmental message may also include a message of petrochemical equivalence. Many renewable, naturally occurring, or non-petroleum derived materials may be known. However, these materials often lack the performance characteristics that consumers have come to expect when used in conjunction with lotion compositions. Therefore, a message of petroleum equivalence may be necessary to educate consumers that the lotions derived from renewable resources, as described above, exhibit equivalent or better performance characteristics as compared to petroleum derived lotions. A suitable petrochemical equivalence message can include comparisons to absorbent articles and/or lotions that are not derived from a renewable resource. This message conveys both the related environmental message and the message of petrochemical equivalence.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present disclosure. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A lotion comprising:
a) an emollient selected from the group consisting of mineral oil, petrolatum, and mixtures thereof, wherein the emollient is derived from a renewable resource and having a bio-based content of from about 10% to about 100% using ASTM D6866-10, method B; and
b) an immobilizing agent selected from the group consisting of C₁₄-C₆₀ fatty alcohols, C₁₄-C₆₀ fatty acids, C₁₄-C₆₀ fatty alcohol ethoxylates having an average degree of ethoxylation ranging from about 2 to about 110, waxes and mixtures thereof.

2. The lotion of claim 1 further comprising at least one surfactant.

3. The lotion of claim 1, wherein the immobilizing agent comprises C₁₆-C₂₂ fatty alcohols selected from the group consisting of cetyl alcohol, stearyl alcohol, behenyl alcohol, and mixtures thereof.

4. The lotion of claim 2, wherein the at least one surfactant comprises a nonionic surfactant.

5. The lotion of claim 1 further comprising skin care actives selected from the group consisting of vitamins and derivatives thereof; sunscreens; preservatives; anti-acne medicaments; antioxidants; skin soothing and healing; chelators and sequestrants; essential oils, skin sensates, proton donating agents; protease and enzyme inhibitors; antimicrobials; humectants; multi-functional agents, and mixtures thereof.

6. The lotion of claim 1, wherein the emollient comprises a bio-based content of from about 30% to about 90% using ASTM D6866-10, method B.

7. The lotion of claim 1, wherein the emollient has an Oil Stability Index of at least about 10 hours.

8. The lotion of claim 1 having a penetration hardness of from about 5 millimeters to about 365 millimeters at 25° C.

9. The lotion of claim 1 having an elastic modulus of from about 5 dynes/cm² to about 50,000 dynes/cm².

10. The lotion of claim 1 having a zero shear viscosity at about 20° C. between about 1.0×10⁶ centipoise and about 1.0×10⁸ centipoise.

11. An absorbent article comprising the lotion of claim 1.

12. A lotion having a bio-based content of from about 10% to about 100% using ASTM D6866-10, method B, and comprising:
a) an emollient selected from the group consisting of natural fats, natural oils, mineral oil, petrolatum, and mixtures thereof, wherein the emollient has an Oil Stability Index of at least about 10 hours; and
b) an immobilizing agent selected from the group consisting of C₁₄-C₆₀ fatty alcohols, C₁₄-C₆₀ fatty acids, C₁₄-C₆₀ fatty alcohol ethoxylates having an average degree of ethoxylation ranging from about 2 to about 110, waxes and mixtures thereof.

13. The lotion of claim 12 further comprising at least one surfactant.

14. The lotion of claim 12, wherein the emollient has an Oil Stability Index of at least about 14 hours.

15. The lotion of claim 12 further comprising skin care actives selected from the group consisting of vitamins and derivatives thereof; sunscreens; preservatives; anti-acne medicaments; antioxidants; skin soothing and healing; chelators and sequestrants; essential oils, skin sensates, proton donating agents; protease and enzyme inhibitors; antimicrobials; humectants; multi-functional agents, and mixtures thereof.

16. The lotion of claim 12 having a penetration hardness of from about 5 millimeters to about 365 millimeters at 25° C.

17. The lotion of claim 12 having an elastic modulus of from about 5 dynes/cm² to about 25,000 dynes/cm².

18. The lotion of claim 12 having a zero shear viscosity at about 20° C. between about 5.0×10⁶ centipoise and about 5.0×10⁷ centipoise.

19. An absorbent article comprising:
a) a topsheet;
b) a lotion applied to at least a portion of the topsheet, wherein the lotion comprises an emollient having an Oil Stability Index of at least 10 hours, and wherein the lotion has a bio-based content of from about 10% to about 100% using ASTM D6866-10, method B;
c) a backsheet; and
d) an absorbent core disposed between the topsheet and the backsheet.

20. The absorbent article of claim 19 having a flexure-resistance of less than about 250 grams.

21. The absorbent article of claim 19, wherein the emollient is selected from the group consisting of natural fats, natural oils, mineral oil, petrolatum, and mixtures thereof.

22. The absorbent article of claim 19, wherein the lotion comprises a penetration hardness of from about 5 millimeters to about 365 millimeters at 25° C.

23. The absorbent article of claim 19, wherein the lotion comprises an elastic modulus of from about 5 dynes/cm² to about 25,000 dynes/cm².

24. The absorbent article of claim 19, wherein the lotion comprises a zero shear viscosity at about 20° C. between about 5.0×10⁶ centipoise and about 5.0×10⁷ centipoise.

25. The absorbent article of claim 19, wherein the emollient comprises from about 30% to about 90% petrolatum, by weight.

26. The absorbent article of claim 19, wherein the lotion further comprises an immobilizing agent.

27. The absorbent article of claim 26, wherein the immobilizing agent is selected from the group consisting of C₁₄-C₆₀ fatty alcohols, C₁₄-C₆₀ fatty acids, C₁₄-C₆₀ fatty alcohol ethoxylates having an average degree of ethoxylation ranging from about 2 to about 110, waxes and mixtures thereof.

28. The absorbent article of claim 19, wherein the lotion further comprises at least one surfactant.

29. The absorbent article of claim 19 comprises an average caliper of less than about 10 mm and a free absorbent capacity of from about 4 to about 125 grams per gram.

30. The absorbent article of claim 19 comprising from about 2 mg to about 300 mg of the lotion.

31. The absorbent article of claim 19, wherein the portion of the topsheet comprising the lotion comprises from about 2 gsm to about 100 gsm of the lotion.
